(19) United States
(12) Patent Application Publication
(10) Pub. No.: US 2026/0253711 A1
(43) Pub. Date: Aug. 27, 2026

(54) System and Method for Generating Adaptive Personalized Therapeutic Protocols Based on Physiological Biomarkers

(71) Applicant: Médéric Degoy, Perpignan (FR)

(72) Inventor: Médéric Degoy, Perpignan (FR)

(21) Appl. No.: 19/546,476

(22) Filed: Feb. 23, 2026

Related U.S. Application Data
(60) Provisional application No. 63/762,661, filed on Feb. 25, 2025.

(51) Int. Cl. G16H 20/70; G16H 10/60; G16H 20/30; G16H 50/70

(52) U.S. Cl. CPC G16H 20/70; G16H 10/60; G16H 20/30; G16H 50/70

(57) ABSTRACT

A method and system generate personalized therapeutic protocols adapted to a user's physiological state. Biomarkers derived from ECG data are used to identify priorities and resources, then select and combine elements from libraries of over four thousand custom voice recordings, ten thousand audio frequency components, ten thousand micro-current frequency profiles, visual elements and textual information. Protocols adapt daily via biofeedback, incorporating guided sessions or frequency-only harmonic boosts, with sound techniques (binaural beats, noises) and optional professional/AI customization over a 21-day cycle for coherence restoration.

### CROSS-REFERENCE TO RELATED APPLICATIONS

This application claims the benefit of U.S. Provisional Patent Application No. 63/762,661, filed Feb. 25, 2025, the entire contents of which are incorporated herein by reference.

### BACKGROUND OF THE INVENTION

### Field of the Invention

The present invention relates generally to personalized wellness and biofeedback systems. More particularly, it pertains to methods and systems for generating adaptive audio-based, information-based, visual-based and micro-current-based therapeutic protocols using physiological biomarkers derived from electrocardiogram (ECG) or similar data, with dynamic daily adjustment for coherence restoration and health optimization.

### Description of Related Art

Conventional meditation, micro-current and biofeedback applications typically use static or minimally adaptive content, such as pre-recorded guided sessions, pre-recorded frequency plans or basic heart rate variability (HRV) feedback. These approaches often lack deep personalization, relying on generic scripts or simple metrics without accounting for multi-dimensional physiological-psychological states or interdependencies. Existing systems may incorporate, for instance, limited audio elements (e.g., binaural beats or ambient sounds) but do not dynamically sequence content based on evolving user biomarkers, priorities, resources, or real-time biofeedback. There remains a need for systems that generate and adapt therapeutic protocols on a daily basis using a large library of custom voice, visual, information and frequency components, informed by comprehensive biomarker analysis and relational mapping, to achieve targeted coherence and wellness outcomes.

### Brief Summary of the Invention

The present invention provides a computer-implemented method and system for generating adaptive personalized therapeutic protocols based on physiological biomarkers. Biomarkers derived from ECG data are used to identify priorities and resources, then select and combine elements from libraries of over four thousand custom voice recordings, ten thousand audio frequency components, thousands of textual and graphic informations, and ten thousand micro-current frequency components. Protocols adapt daily via biofeedback, incorporating guided sessions (“Personal Guide”) or frequency-only harmonic boosts, with sound techniques (binaural beats, background noises) and optional professional/AI customization over a 21-day cycle for coherence restoration. Optional synergistic delivery via audio and micro-current devices (“Harmonizer”) applies custom frequencies through both channels for enhanced physiological effect. Extensive information on each biomarker is also given for deep understanding of the needed changes.

### DETAILED DESCRIPTION

The invention is illustrated by way of example and not limitation in the figures of the accompanying drawings, in which like reference numerals indicate similar elements. In particular:
100 designates the Biomarkers Input
110 designates the Priority/Resource Identification
120 designates the Selection/Combination (with resource-first logic)
130 designates the obtained list of priorities and resources
135 designates the professional or AI intervention
140 designates the Library Access (Voices/Frequencies)
150 designates the Daily Adaptation (21-day cycle)
160 designates the Output Modes choice
170 designates the Guided output mode
175 designates the Harmonic Boost output mode
180 designates the Harmonizer micro-current output mode
190 designates the protocol output
200 designates the daily selection algorithm
210 designates the reevaluation
220 designates the charts and evolution visualization
300 designates the list of items for training
310 designates an individual item to train
320 designates the training information sent to the user (e.g., breathing, attention, or exercise guidance)
330 designates the live biofeedback measurement
340 designates the live adaptation of the training protocol
350 designates the real-time result visualization/displayed to the user

This invention provides a system and method for generating adaptive personalized therapeutic protocols (190) based on physiological biomarkers derived from electrocardiogram (ECG) analysis (100). The system uses over 1,000 novel biomarkers—each linked to specific structures in energy, body, or mind—and their relational mapping via systems theory to inform precise content selection (130) and sequencing (150).

Biomarkers are generated via advanced signal processing and proprietary algorithms, capturing multi-dimensional states (e.g., organ function, autonomic balance, energetic flux, coherence, entropy). Systems theory maps causal relationships, feedback loops, resource-nourishing influences, and priority elements, (110) enabling targeted personalization (150).

Daily therapeutic protocols are constructed from a library exceeding 4,000 custom voice elements (“Personal Guide”), 10,000 audio frequency components (“Harmonic Boost”), thousands of textual and graphic informations, and 10,000 micro-current frequency components (140). Selection and combination prioritize resources (strongest nourishing elements) first (120), then address priorities (areas needing attention/root causes) (130), with daily adaptation via biofeedback and re-evaluations (210). Additional sound techniques (background noises, binaural beats, specific frequencies) (175) and micro-currents (harmonic specific electrical frequencies delivered to the body at specific places in a synergetic way) (180) maximize efficiency. User can choose individual or combinations of the various modes (160). The program follows a 21-day structure, with evolving priorities and daily playing items updated progressively for habit reprogramming and coherence restoration (200).

Biomarkers and relationships are visualized through graphics, body maps, and unique representations (reflecting energy, coherence, entropy, attention, intention, horizon, links) (220), supporting dynamic reassessments and changes in daily priorities/meditation content (200).

Professionals or AI tools can customize protocols, create new therapeutic items (integrable into user sessions), and refine based on relational data (135).

Protocols adapt dynamically, with outputs including guided sessions (170), frequency-only modes (175), or synergistic delivery via audio, visuals and optional micro-current devices” (180), where custom frequencies are applied through both channels in coordinated fashion for enhanced physiological effect.

The system supports real-time training of selected items from a list (300). An individual item (310) is chosen and trained through multisensory output (160) including guided sessions (170), frequency-only modes (175), or synergistic delivery via audio, visuals and optional micro-current devices (180). Training information (320), such as breathing guidance, attention exercises, or other targeted protocols, is presented to the user. Live biofeedback measurement (via ECG sensor or other device) (330) assesses immediate physiological response, enabling real-time adaptation of the training protocol (340) to optimize toward specific targets (e.g., enhanced coherence, reduced entropy). The result is visualized and displayed to the user in real time (350) for immediate feedback and progress tracking.

The invention may be embodied in wearable devices, software, and cloud systems, delivering audio, visual, interactive, and optional micro-current outputs (190) optimized by systems theory for wellness, stress management, and coherence restoration.

### BRIEF DESCRIPTION OF THE DRAWINGS

FIG. 1 is a block diagram illustrating an exemplary system for generating adaptive personalized therapeutic protocols, showing the flow from biomarker input through priority/resource identification, selection and combination of library elements, optional modification by a professional or AI, adaptation over a 21-day cycle, and delivery via multiple output modes including guided audio, harmonic boost, visuals, information and synergistic micro-current.

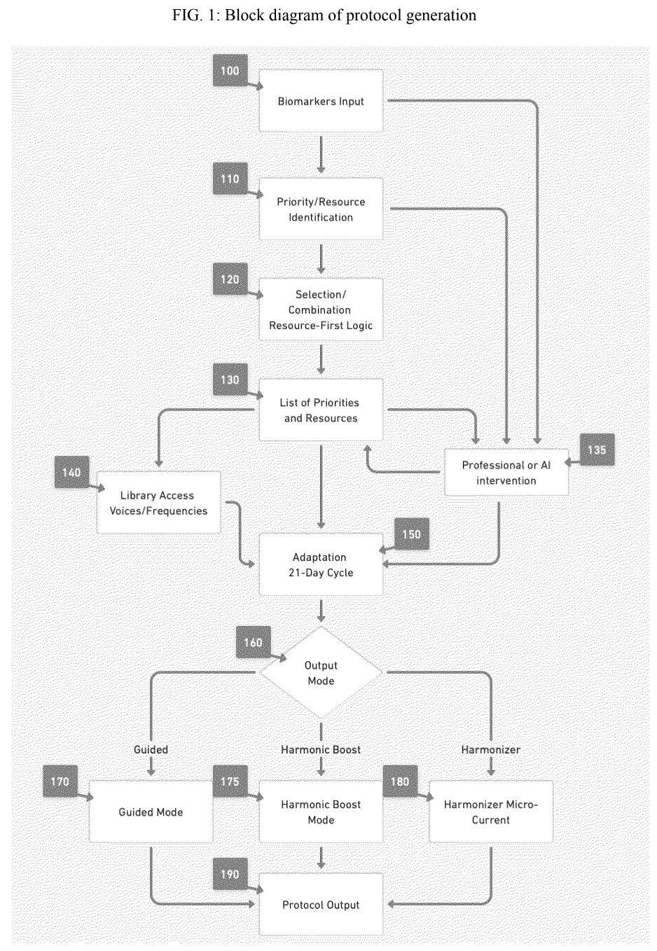

FIG. 2 is a flowchart depicting an exemplary process for the therapeutic protocol adaptation cycle, including daily biomarker-driven selection, reevaluation, and visualization of charts and evolution over time.

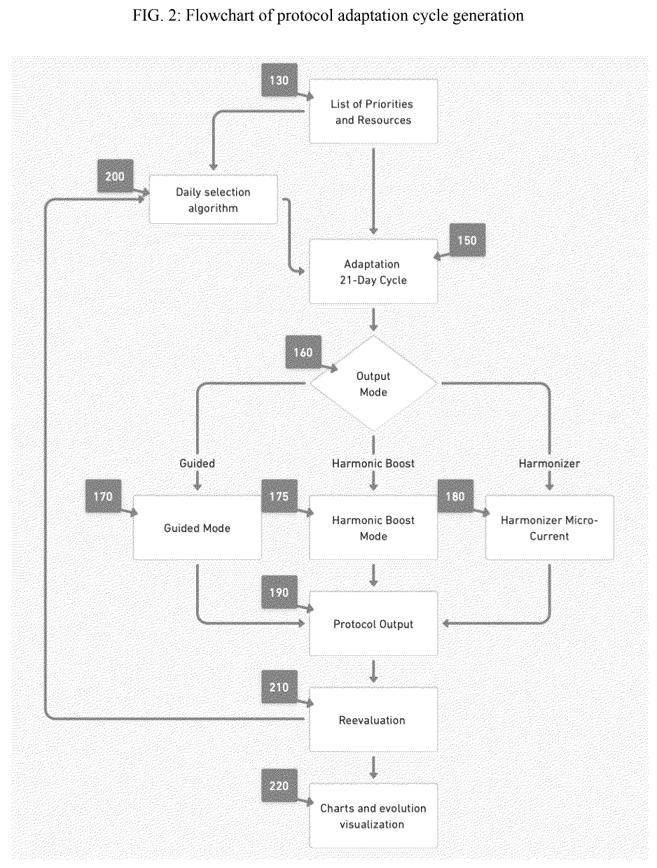

FIG. 3 is a flowchart depicting a multisensory synchronization and real-time training loop, including a list of items for training, selection of individual items, protocol output with guided sessions, frequency-only modes, and synergistic audio/visuals/micro-current delivery, delivery of training information (e.g., breathing, attention, exercises), live biofeedback measurement, real-time protocol adaptation, and result visualization.

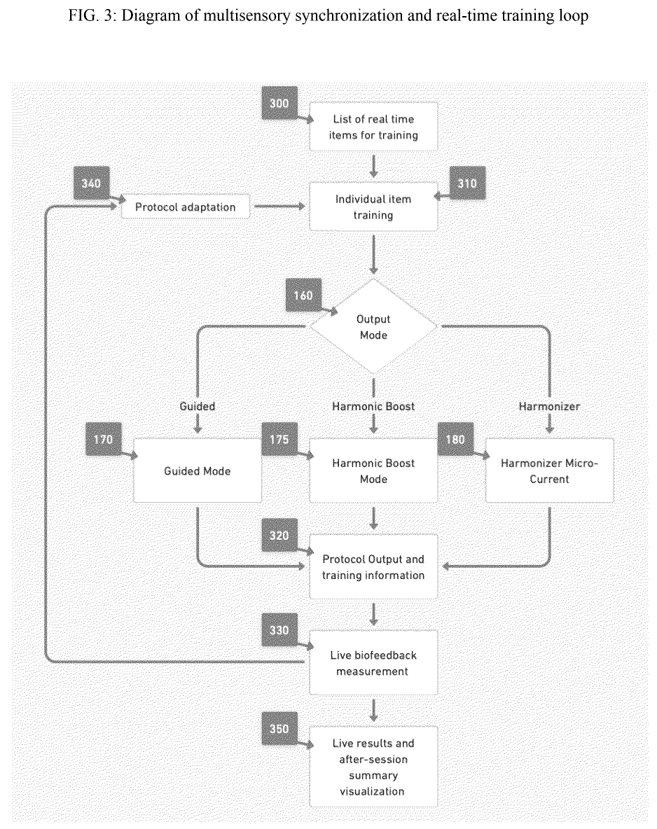

1. A computer-implemented method for generating a personalized therapeutic protocol, comprising: obtaining a current set of physiological biomarkers associated with a user, the biomarkers derived from electrocardiogram data; accessing a digital library comprising more than four thousand pre-recorded custom voice elements, more than ten thousand distinct audio frequency components, various textual and visual informations and more than ten thousand distinct micro-current frequency components; identifying priorities (areas requiring attention or root causes) and resources (strong nourishing or supportive elements) based on the biomarkers and their relational mapping; selecting and combining a subset of the voice elements, informations, visual and frequency components, prioritizing resources before addressing priorities; generating a therapeutic protocol that includes at least one of a guided session incorporating selected voice elements, audio frequency-only harmonic boost session, micro-current-only frequency session, textual informations and visuals; and delivering the protocol to the user via an output device, with daily adaptation informed by biofeedback.

2. The method of claim 1, wherein the daily adaptation rule modifies selection and combination in response to detected changes in at least one biomarker over a twenty-four hour period or re-evaluation.

3. The method of claim 1, further comprising incorporating additional sound techniques selected from background noises, binaural beats, specific audio frequencies, or harmonic progressions to enhance coherence restoration.

4. The method of claim 1, wherein the protocol follows a structured 21-day cycle, with evolving priorities and therapeutic items updated progressively based on biomarker trends and re-evaluations.

5. The method of claim 1, further comprising receiving input from a professional operator or AI customization tool to create new therapeutic items or modify protocol components.

6. The method of claim 1, wherein the biomarkers include multi-dimensional indicators of energy flux, coherence, entropy, attention, intention, horizon, and links, used to sequence protocol elements.

7. The method of claim 1, wherein the relational mapping informs prioritization of resources for initial protocol sections, followed by targeted intervention on priorities.

8. The method of claim 1, wherein the protocol includes dynamic reassessment of biomarkers leading to changes in daily priorities and therapeutic content.

9. The method of claim 1, wherein the audio meditation protocol is delivered via a synergistic dual-mode system, applying custom frequencies through both audio output and micro-current stimulation to a user for enhanced physiological effect.

10. A system for delivering adaptive therapeutic content, comprising: a processor; memory storing a library of over four thousand voice elements and over ten thousand frequency components; instructions that, when executed, cause the processor to obtain physiological biomarkers from ECG data, identify priorities and resources, select/combine elements prioritizing resources, generate a protocol with guided or harmonic boost modes, and deliver it via audio output, with daily adaptation.

11. The system of claim 10, further comprising a biofeedback module for real-time adjustment based on user response.

12. The system of claim 10, further comprising a micro-current delivery interface (“Harmonizer”) for applying custom frequencies in synergy with audio output.

13. A computer-implemented method for synergistic delivery of personalized therapeutic frequencies, comprising: obtaining physiological biomarkers from a user; identifying custom frequencies based on the biomarkers and relational mapping; generating a protocol that applies the custom frequencies via at least one of audio output (harmonic boost), micro-current stimulation (Harmonizer device), informations and visuals, or part or all simultaneously in coordinated fashion; delivering the frequencies and informations to the user to promote coherence restoration or wellness; and adapting the delivery daily based on biofeedback.

14. The method of claim 13, wherein the micro-current delivery uses low-level electrical signals synchronized with audio frequencies for synergistic effect.

15. The method of claim 1, wherein the system generates visual outputs in real-time synergy with the audio protocol, the visuals comprising at least one of: textual informations; colors computed from audio frequency components to create visual harmony with the sound; or computer-generated art and shapes dynamically altered in real-time based on the biomarker-derived audio frequencies, thereby providing multisensory coherence reinforcement.

16. The method of claim 15, further comprising a real-time training mode that simultaneously emits personalized audio frequencies, dynamic visuals, and optional micro-current stimulation while measuring user physiological response via biofeedback, the system iteratively adjusting at least one emitted modality to train a targeted physiological or psychological state, such as improved breathing patterns, attention focus, or coherence levels.

17. The method of claim 1, further comprising exporting the generated audio protocol as a custom digital audio and/or video file, such as an MP3 containing the selected harmonic boost frequency content, voice-guided elements, or combined modalities, the file formatted for offline playback or sharing while preserving the personalized biomarker-derived structure, or a video file with additional synchronized visual elements.

18. The method of claim 1, wherein new therapeutic items are created or selected for integration into the protocol based on a predicted or measured impact on specific biomarkers, the selection process evaluating each item's effect on at least one of coherence, energy flux, entropy, or relational mapping parameters.

19. The method of claim 18, wherein the impact assessment of new items is determined by at least one of: pre-computed metadata associated with each item indicating its influence on biomarker categories; real-time biofeedback testing during a trial application of the item; or AI-driven prediction using the relational mapping of biomarkers to forecast coherence restoration outcomes.

20. The method of claim 1, wherein the protocol delivery includes multisensory synchronization across audio, visual, and optional micro-current channels, the synchronization dynamically adjusted based on real-time biofeedback to optimize user coherence and training efficacy.
